(19) United States
(12) Patent Application Publication
(10) Pub. No.: US 2009/0054512 A1
(43) Pub. Date: Feb. 26, 2009

(54) USE OF ORGANIC COMPOUNDS

(76) Inventor: James E. Foley, Sparta, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/158,772

(22) PCT Filed: Jan. 4, 2007
(86) PCT No.: PCT/US07/60081

§ 371 Date: Jun. 23, 2008

Related U.S. Application Data
(60) Provisional application No. 60/757,051, filed on Jan. 6, 2006.
(60) Provisional application No. 60/772,655, filed on Feb. 13, 2006.

(51) Int. Cl. A61K 31/40; A61P 3/00

(52) U.S. Cl. 514/423

(57) ABSTRACT

A method for improving glucose control by administering a DPP-IV inhibitor to a patient in need thereof, before or with the evening meal.

The invention relates to a method for treating a patient suffering from hyperglycemia wherein vildagliptin, preferably 100 mg, 50 mg or 25 mg of vildagliptin, or a salt thereof is administered to said patient before or with the evening meal, or to a method to reduce the overnight hepatic glucose production in a patient suffering from hyperglycemia, wherein vildagliptin, preferably 100 mg, 50 mg or 25 of vildagliptin, or a salt thereof is administered to said patient before or with the evening meal. Preferably to a patient with type 2 diabetes and with baseline glycosylated hemoglobin (HbA1c)>8.0%.

The treated patients are preferably suffering from hyperglycemia such as diabetes mellitus preferably non-insulin-dependent diabetes mellitus or Impaired Glucose Metabolism (IGM) preferably Impaired Glucose Tolerance (IGT).

Diabetes mellitus is a relatively common disorder which is characterized by hyperglycemia. There are three basic types of diabetes mellitus, type I or insulin-dependent diabetes mellitus (IDDM), type 2 or non-insulin-dependent diabetes mellitus (NIDDM), and type A insulin resistance. Patients with either type I or type 2 diabetes can become insensitive to the effects of exogenous insulin (“insulin resistant”) through a variety of mechanisms. Type A insulin resistance results from either mutations in the insulin receptor gene or defects in post-receptor sites of action critical for glucose metabolism. Diabetes is generally controlled through administration of exogenous insulin (especially in type I diabetics), dietary control and exercise (especially in type 2 diabetics) or both.

Impaired Glucose Metabolism (IGM) is defined by blood glucose levels that are above the normal range but are not high enough to meet the diagnostic criteria for type 2 diabetes mellitus. The incidence of IGM varies from country to country, but usually occurs 2-3 times more frequently than overt diabetes. Until recently, individuals with IGM were felt to be pre-diabetics, but data from several epidemiologic studies argue that subjects with IGM are heterogeneous with respect to their risk of diabetes and their risk of cardiovascular morbidity and mortality. Among subjects with IGM, about 58% have Impaired Glucose Tolerance (IGT), another 29% have Impaired Fasting Glucose (IFG), and 13% have both abnormalities (IFG/IGT). IGT is characterized by elevated postprandial (post-meal) hyperglycemia while IFG has been defined by the ADA on the basis of fasting glycemic values.

The categories of Normal Glucose Tolerance (NGT), IGM and type 2 diabetes mellitus were defined by the ADA (American Diabetes Association) in 1997.

The use of DPP-4 inhibitors for the treatment of hyperglycemia is a promising new therapeutic approach.

DPP-4 inhibitors increase post-meal plasma levels of intact (active) GLP-1 and GIP in patients with hyperglycemia e.g. type 2 diabetes mellitus by inhibiting DPP-4, the enzyme that degrades and inactivates these incretin hormones. Because incretin hormone release is dependent on nutrient ingestion, it might be expected that DPP-4 inhibition would have little influence on GLP-1 or GIP in the fasting state.

During the clinical development of DPP-4 inhibitors, the applicant has surprisingly discovered a method of treatment which can provide additional therapeutic benefits for the treatment of hypoglycemic patient.

Indeed, additional therapeutic benefits (e.g. pharmacodynamic benefits) can be shown if the DPP-4 inhibitor is administered to the patient before or with the evening meal, preferably to a patient with type 2 diabetes and with baseline HbA1c>8.0%.

Thus the present invention concerns;

A method for treating a patient suffering from hyperglycemia, wherein a DPP-4 inhibitor or a salt thereof is administered to said patient before or with the evening meal.

Use of a DPP-4 inhibitor or a salt thereof for the manufacture of a medicament for the treatment of hyperglycemia, wherein the DPP-4 inhibitor is administered to said patient before or with the evening meal.

A method to reduce the overnight hepatic glucose production in a patient suffering from hyperglycemia, wherein a DPP-4 inhibitor or a salt thereof is administered to said patient before or with the evening meal.

Use of a DPP-4 inhibitor or a salt thereof for the manufacture of a medicament to reduce the overnight hepatic glucose production in a patient suffering from hyperglycemia, wherein the DPP-4 inhibitor is administered to said patient before or with the evening meal.

The herein described methods or uses wherein;
the patient is suffering from type 2 diabetes, or
the patient is suffering from type 2 diabetes with baseline HbA1c>8.0%.

The herein described methods or uses wherein;
the patient is suffering from IGT.

The herein described methods or uses wherein;
the DPP-4 inhibitor is vildagliptin or a salt thereof,
between 25 mg and 100 mg or 50 mg and 100 mg of vildagliptin or a salt thereof, is administered before or with the evening meal, or
25 mg, 50 mg or 100 mg of vildagliptin or a salt thereof, is administered before or with the evening meal.

A method for treating a patient with type 2 diabetes and with baseline HbA1c>8.0%, wherein between 25 mg and 100 mg or 50 mg and 100 mg of vildagliptin (preferably 25, 50 or 100 mg of vildagliptin) or a salt thereof is administered to said patient before or with the evening meal.

Use of a DPP-4 inhibitor or a salt thereof for the manufacture of a medicament for the treatment of a patient with type 2 diabetes and with baseline HbA1c>8.0%, wherein between 25 mg and 100 mg or 50 mg and 100 mg of vildagliptin (preferably 25, 50 or 100 mg of vildagliptin) or a salt thereof is administered to said patient before or with the evening meal.

A method to reduce the overnight hepatic glucose production in a patient with type 2 diabetes and with baseline HbA1c>8.0%, wherein between 100 mg and 50 mg of vildagliptin (preferably 25, 50 or 100 mg of vildagliptin) or a salt thereof is administered to said patient before or with the evening meal.

Use of a DPP-4 inhibitor or a salt thereof for the manufacture of a medicament to reduce the overnight hepatic glucose production in a patient with type 2 diabetes and with baseline HbA1c>8.0%, wherein between 25 mg and 100 mg or 50 mg and 100 mg of vildagliptin (preferably 25, 50 or 100 mg of vildagliptin) or a salt thereof is administered to said patient before or with the evening meal.

The herein described methods or uses wherein a daily dosage of 100 mg of vildagliptin or a salt thereof, is administered to the patient.

The herein described methods or uses wherein a daily single dosage of 100 mg of vildagliptin or a salt thereof, is administered to the patient, i.e. 100 mg is administered before or with the evening meal.

The herein described methods or uses wherein 50 mg bid (twice daily i.e. 100 mg per day) of vildagliptin or a salt thereof, are administered and 50 mg are administered to the patient before or with the evening meal. Preferably the first 50 mg are administered before (e.g. up to 1 hours before, or up to 10 minutes before) the breakfast or with the breakfast.

The herein described methods or uses wherein 25 mg bid (twice daily i.e. 50 mg per day) of vildagliptin or a salt thereof, are administered and 25 mg are administered to the patient before or with the evening meal. Preferably the first 25 mg are administered before (e.g. up to 1 hours before, or up to 10 minutes before) the breakfast or with the breakfast.

The herein described methods or uses wherein vildagliptin or a salt thereof is administered about 30 minutes prior to the dinner meal.

The applicant has also discovered that the overnight hepatic glucose production can be reduced in a patient with type 2 diabetes especially with a baseline HbA1c>8.0%, if at least a dosage of 100 mg of vildagliptin or a salt thereof, is administered to the patient before or with the morning meal, or before or with the lunch.

Thus, in a further aspect, the present invention concerns a method to reduce the overnight hepatic glucose production in a patient with type 2 diabetes especially with a baseline HbA1c>8.0%, wherein at least 100 mg of vildagliptin (preferably between 100 mg and 150 mg or a specific dosage of 100 mg or 150 mg of vildagliptin) or a salt thereof is administered to said patient before or with the morning meal, or before or with the lunch.

Thus, in a further aspect, the present invention concerns the use of vildagliptin or a salt thereof for the manufacture of a medicament to reduce the overnight hepatic glucose production in a patient with type 2 diabetes especially with a baseline HbA1c>8.0%, wherein at least 100 mg of vildagliptin (preferably between 100 mg and 150 mg or a specific dosage of 100 mg or 150 mg of vildagliptin) or a salt thereof is administered to said patient before or with the morning meal, or before or with the lunch.

The herein described methods or uses wherein the DPP-4 inhibitor is administered in combination with metformin. Preferably with 250 mg, 500 mg, 750 mg, 850 mg or 1000 mg of metformin. Preferably metformin is also administered before or with the evening meal.

The term “DPP-IV inhibitor” is intended to indicate a molecule that exhibits inhibition of the enzymatic activity of DPP-IV and functionally related enzymes, such as from 1-100% inhibition, and specially preserves the action of substrate molecules, including but not limited to glucagon-like peptide-1, gastric inhibitory polypeptide, peptide histidine methionine, substance P, neuropeptide Y, and other molecules typically containing alanine or proline residues in the second aminoterminal position. Treatment with DPP-IV inhibitors prolongs the duration of action of peptide substrates and increases levels of their intact, undegraded forms leading to a spectrum of biological activities relevant to the disclosed invention.

DPP-IV can be used in the control of glucose metabolism because its substrates include the insulinotropic hormones Glucagon like peptide-1 (GLP-1) and Gastric inhibitory peptide (GIP). GLP-1 and GIP are active only in their intact forms; removal of their two N-terminal amino acids inactivates them. In vivo administration of synthetic inhibitors of DPP-IV prevents N-terminal degradation of GLP-1 and GIP, resulting in higher plasma concentrations of these hormones, increased insulin secretion and, therefore, improved glucose tolerance. For that purpose, chemical compounds are tested for their ability to inhibit the enzyme activity of purified CD26/DPP-IV. Briefly, the activity of CD26/DPP-IV is measured in vitro by its ability to cleave the synthetic substrate Gly-Pro-p-nitroanilide (Gly-Pro-pNA). Cleavage of Gly-Pro-pNA by DPP-IV liberates the product p-nitroanilide (pNA), whose rate of appearance is directly proportional to the enzyme activity. Inhibition of the enzyme activity by specific enzyme inhibitors slows down the generation of pNA. Stronger interaction between an inhibitor and the enzyme results in a slower rate of generation of pNA. Thus, the degree of inhibition of the rate of accumulation of pNA is a direct measure of the strength of enzyme inhibition. The accumulation of pNA is measured with a spectrophotometer. The inhibition constant, Ki, for each compound is determined by incubating fixed amounts of enzyme with several different concentrations of inhibitor and substrate.

In the present context “a DPP-IV inhibitor” is also intended to comprise active metabolites and prodrugs thereof, such as active metabolites and prodrugs of DPP-IV inhibitors. A “metabolite” is an active derivative of a DPP-IV inhibitor produced when the DPP-IV inhibitor is metabolised. A “prodrug” is a compound that is either metabolised to a DPP-IV inhibitor or is metabolised to the same metabolite(s) as a DPP-IV inhibitor. In the present context the term “a DPP-IV inhibitor” is also intended to comprise pharmaceutical salts thereof.

DPP-IV inhibitors are known in the art. In the following reference is made to representatives of DPP-IV inhibitors:

Preferred DPP-IV inhibitors are described in the following patent applications; WO 02053548 especially compounds 1001 to 1293 and examples 1 to 124, WO 02067918 especially compounds 1000 to 1278 and 2001 to 2159, WO 02066627 especially the described examples, WO 02/068420 especially all the compounds specifically listed in the examples I to LXIII and the described corresponding analogues, even preferred compounds are 2(28), 2(88), 2(119), 2(136) described in the table reporting IC50, WO 02083128 such as in the claims 1 to 5 especially compounds described in examples 1 to 13 and the claims 6 to 10, US 2003096846 especially the specifically described compounds, WO 2004/037181 especially examples 1 to 33, WO 0168603 especially compounds of examples 1 to 109, EP1258480 especially compounds of examples 1 to 60, WO 0181337 especially examples 1 to 118, WO 02083109 especially examples 1A to 1D, WO 030003250 especially compounds of examples 1 to 166, most preferably 1 to 8, WO 03035067 especially the compounds described in the examples, WO 03/035057 especially the compounds described in the examples, US2003216450 especially examples 1 to 450, WO 99/46272 especially compounds of claims 12, 14, 15 and 17, WO 0197808 especially compounds of claim 2, WO 03002553 especially compounds of examples 1 to 33, WO 01/34594 especially the compounds described in the examples 1 to 4, WO 02051836 especially examples 1 to 712, EP1245568 especially examples 1 to 7, EP1258476 especially examples 1 to 32, US 2003087950 especially the described examples, WO 02/076450 especially examples 1 to 128, WO 03000180 especially examples 1 to 162, WO 03000181 especially examples 1 to 66, WO 03004498 especially examples 1 to 33, WO 0302942 especially examples 1 to 68, U.S. Pat. No. 6,482,844 especially the described examples, WO 0155105 especially the compounds listed in the examples 1 and 2, WO 0202560 especially examples 1 to 166, WO 03.004496 especially examples 1 to 103, WO 03/024965 especially examples 1 to 54, WO 0303727 especially examples 1 to 209, WO 0368757 especially examples 1 to 88, WO 03074500 especially examples 1 to 72, examples 4.1 to 4.23, examples 5.1 to 5.10, examples 6.1 to 6.30, examples 7.1 to 7.23, examples 8.1 to 8.10, examples 9.1 to 9.30, WO 02038541 especially examples 1 to 53, WO 02062764 especially examples 1 to 293, preferably the compound of example 95 (2-{{3-(Aminomethyl)-4-butoxy-2-neopentyl-1-oxo-1,2 dihydro-6-isoquinolinyl}oxy}acetamide hydrochloride), WO 02308090 especially examples 1-1 to 1-109, examples 2-1 to 2-9, example 3, examples 4-1 to 4-19, examples 5-1 to 5-39, examples 6-1 to 6-4, examples 7-1 to 7-10, examples 8-1 to 8-8, examples 7-1 to 7-7 of page 90, examples 8-1 to 8-59 of pages 91 to 95, examples 9-1 to 9-33, examples 10-1 to 10-20, US 2003225102 especially compounds 1 to 115, compounds of examples 1 to 121, preferably compounds a) to z), aa) to az), ba) to bz), ca) to cz) and da) to dk), WO 0214271 especially examples 1 to 320, US 2003096857, U.S. application Ser. No. 09/788,173 filed Feb. 16, 2001 (attorney file LA50) especially the described examples, WO99/38501 especially the described examples, WO99/46272 especially the described examples and DE19616 486 A1 especially val-pyr, val-thiazolidide, isoleucyl-thiazolidide, isoleucyl-pyrrolidide, and fumar salts of isoleucyl-thiazolidide and isoleucyl-pyrrolidide.

Further preferred DPP-IV inhibitors include the specific examples disclosed in U.S. Pat. No. 6,124,305 and U.S. Pat. No. 6,107,317, International Patent Applications, Publication Numbers WO 9819998, WO 95153 09 and WO 9818763; such as 1[2-[(5 eyanopyridin-2-yl)aminoethylamino]acetyl-2-cyano-(S)-pyrrolidine and (2S)-1-[(2S)-2 amino-3,3-dimethylbutanoyl]-2-pyrrolidinecarbonitrile.

In a further preferred embodiment, the DPP-IV inhibitor is a N-peptidyl-O-aroyl hydroxylamine or a pharmaceutically acceptable salt thereof. Aroyl is, for example, naphthylcarbonyl; or benzoyl which is unsubstituted or mono- or disubstituted, for example, by lower alkoxy, lower alkyl, halogen or, preferably, nitro. The peptidyl moiety comprises preferably two α-amino acids, e.g. glycine, alanine, leucine, phenylalanine, lysine or proline, of which the one attached directly to the hydroxylamine nitrogen atom is preferably proline.

In each case in particular in the compound claims and the final products of the working examples, the subject matter of the final products, the pharmaceutical preparations and the claims are hereby incorporated into the present application by reference to these publications.

WO 9819998 discloses N—(N′-substituted glycyl)-2-cyano pyrrolidines, in particular 1-[2-[5-Cyanopyridin-2-yl]amino]-ethylamino]acetyl-2-cyano-(S)-pyrrolidine.

Preferred compounds described in WO03/002553 are listed on pages 9 to 11 and are incorporated into the present application by reference.

DE19616 486 A1 discloses val-pyr, val-thiazolidide, isoleucyl-thiazolidide, isoleucyl-pyrrolidide, and fumar salts of isoleucyl-thiazolidide and isoleucyl-pyrrolidide.

WO 0034241 and U.S. Pat. No. 6,110,949 disclose N-substituted adamantyl-amino-acetyl-2-cyano pyrrolidines and W (substituted glycyl)-4-cyano pyrrolidines respectively. DPP-IV inhibitors of interest are specially those cited in claims 1 to 4.

WO 9515309 discloses amino acid 2-cyanopyrrolidine amides as inhibitors of DPP-IV and WO 9529691 discloses peptidyl derivates of diesters of alpha-aminoalkylphosphonic acids, particularly those with proline or related structures. DPP-IV inhibitors of interest are specially those cited in Table 1 to 8.

In WO 01/72290 DPP-IV inhibitors of interest are specially those cited in example 1 and claims 1, 4, and 6.

WO 9310127 discloses proline boronic esters useful as DPP-IV inhibitors. DPP-IV inhibitors of interest are specially those cited in examples 1 to 19.

Published patent application WO 9925719 discloses sulphostin, a DPP-IV inhibitor prepared by culturing a Streptomyces microorganism.

WO 9938501 discloses N-substituted 4- to 8-membered heterocyclic rings. DPP-IV inhibitors of interest are specially those cited in claims 15 to 20.

WO 9946272 discloses phosphoric compounds as inhibitors of DPP-IV. DPP-IV inhibitors of interest are specially those cited in claims 1 to 23.

Other preferred DPP-IV inhibitors are the compounds of formula I, II or III disclosed in the patent application WO 03/057200 on page 14 to 27. Most preferred DPP-IV inhibitors are the compounds specifically described on pages 28 and 29.

Published patent applications WO 9967278 and WO 9967279 disclose DPP-IV prodrugs and inhibitors of the form A-B-C where C is either a stable or unstable inhibitor of DPP-IV.

Preferably, the N-peptidyl-O-aroyl hydroxylamine is a compound of formula VII

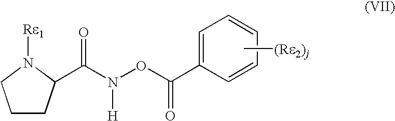

wherein
j is 0, 1 or 2; Rε₁ represents the side chain of a natural amino acid; and Rε₂ represents lower alkoxy, lower alkyl, halogen or nitro; or a pharmaceutically acceptable salt thereof.

In a very preferred embodiment of the invention, the N-peptidyl-O-aroyl hydroxylamine is a compound of formula VIIa

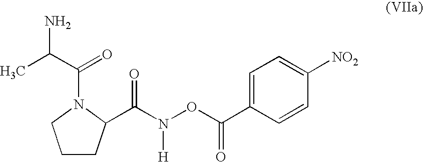

or a pharmaceutically acceptable salt thereof.

N-Peptidyl-O-aroyl hydroxylamines, e.g. of formula VII or VIIa, and their preparation are described by H. U. Demuth et al. in J. Enzyme Inhibition 1988, Vol. 2, pages 129-142, especially on pages 130-132.

Preferred DPP-IV inhibitors are those described by Mona Patel and col. (Expert Opinion Investig Drugs. 2003 April; 12(4):623-33) on the paragraph 5, especially P32/98, K-364, FE-999011, BDPX, NVP-DDP-728 and others, which publication is hereby incorporated by reference especially the described DPP-IV inhibitors.

Another preferred DPP-IV inhibitor is the No. 815541 (T 6666) from Tanabe.

Preferred DPP-IV inhibitors are also described in the patent applications WO 02/083128, especially the compounds described in the examples 1 to 13, U.S. Pat. No. 6,395,767 examples 1 to 109 and WO 03/033671 all the specifically described compounds e.g. compounds 1 to 393, compounds of pages 67-70.

FE-999011 is described in the application WO 95/15309 page 14, as compound No. 18.

Another preferred inhibitor is the compound BMS-477118 disclosed in WO 2001068603 or U.S. Pat. No. 6,395,767 (compound of example 60) also known as is (1S,3S,5S)-2-[(2S)-2-amino-2-(3-hydroxytricyclo[3.3.1.1^{3.7}]dec-1-yl)-1-oxoethyl]-2-azabicyclo[3.1.0]hexane-3-carbonitrile, benzoate (1:1) as depicted in Formula M of the patent application WO 2004/052850 on page 2, and the corresponding free base, (IS,3S,5S)-2-[(2S)-2-amino-2-(3-hydroxy-tricyclo[3.3.1.1^{3.7}]dec-1-yl)-1-oxoethyl]-2-azabicyclo-[3.1.0]hexane-3-carbonitrile (M′) and its monohydrate (M″) as depicted in Formula M of the patent application WO 2004/052850 on page 3. The compound BMS-477118 is also known as saxagliptin.

Another preferred inhibitor is the compound GSK23A disclosed in WO 03/002531 (example 9) also known as (2S,4S)-1-((2R)-2-Amino-3-[(4-methoxybenzyl)sulfonyl]-3-methylbutanoyl)-4-fluoropyrrolidine-2-carbonitrile hydrochloride.

P32/98 (CAS number: 251572-86-8) also known as 3-[(2S,3S)-2-amino-3-methyl-1-oxopentyl]thiazolidine can be used as 3-[(2S,3S)-2-amino-3-methyl-1-oxopentyl]thiazolidine and (2E)-2-butenedioate (2:1) mixture and is described in WO 99/61431 and the below formula,

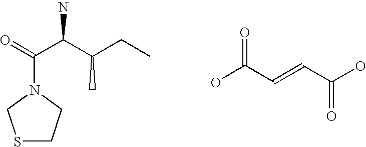

is described in WO 99/61431 and also in Diabetes 1998, 47, 1253-1258, in the name of Probiodrug, as well as the compound P93/01 described by the same company.

Other very preferred DPP-IV inhibitors are the compounds disclosed in the patent application WO 02/083128 such as in the claims 1 to 5. Most preferred DPP-IV inhibitors are the compounds specifically described by the examples 1 to 13 and the claims 6 to 10.

Other very preferred DPP-IV inhibitors are the compounds disclosed By Bristol-Myers Squibb such as Saxagliptin (BMS477118).

Other very preferred DPP-IV inhibitors of the invention are described in the International patent application WO 02/076450 (especially the examples 1 to 128) and by Wallace T. Ashton (Bioorganic & Medicinal Chemistry Letters 14 (2004) 859-863) especially the compound 1 and the compounds listed in the tables 1 and 2. The preferred compound is the compound 21e (table 1) of formula:

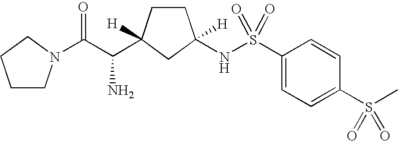

Other preferred DPP-IV inhibitors are described in the patent applications WO 2004/037169 especially those described in the examples 1 to 48 and WO 02/062764 especially the described examples 1 to 293, even preferred are the compounds 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide described on page 7 and also in the patent application WO2004/024184 especially in the reference examples 1 to 4.

Other preferred DPP-IV inhibitors are described in the patent application WO 03/004498 especially examples 1 to 33 and most preferably the compound of the formula

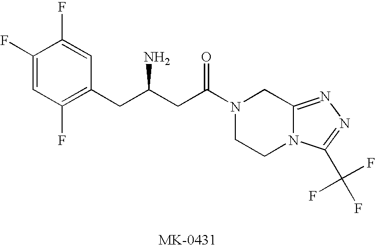

described by the example 7 and also known as MK-0431 or Sitagliptin (INN).

In each case in particular in the compound claims and the final products of the working examples, the subject matter of the final products, the pharmaceutical preparations and the claims are hereby incorporated into the present application by reference to these publications.

Preferred DPP-IV inhibitors are also described in the application WO 2004/037181 especially examples 1 to 33 and most preferably the compounds described in the claims 3 to 5.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl)amino]ethylamino]acetyl-2-cyano-(S)-pyrrolidine, MK-431 and pharmaceutical salts thereof.

Most preferred DPP-IV inhibitors are selected from [S]-1-[2-(5-cyano-2-pyridinylamino)ethylamino]acetyl-2-pyrrolidine carbonitrile monohydrochloride, (S)-1-[(3-hydroxy-1-adamantyl)amino]acetyl-2-cyano-pyrrolidine and L-threo-isoleucyl thiazolidine (compound code according to Probiodrug: P32/98 as described above), MK-0431, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally pharmaceutical salts thereof.

[S]-1-[2-(5-cyano-2-pyridinylamino)ethylamino]acetyl-2-pyrrolidine carbonitrile monohydrochloride and (S)-1-[(3-hydroxy-1-adamantyl)amino]acetyl-2-cyano-pyrrolidine are specifically disclosed in Example 3 of WO 98/19998 and Example 1 of WO 00/34241, respectively.

Especially preferred are 1-{2-[(5-cyanopyridin-2-yl)amino]ethylamino}acetyl-2-(S)-cyanopyrrolidine (also named [S]-1-[2-(5-cyano-2-pyridinylamino)ethylamino]acetyl-2-pyrrolidine carbonitrile monohydrochloride or DPP728), of formula:

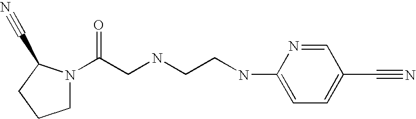

especially the dihydrochloride and monohydrochloride form thereof, pyrrolidine, 1-[(3-hydroxy-1-adamantyl)amino]acetyl-2-cyano-, (S) (also named (S)-1-[(3-hydroxy-1-adamantyl)amino]acetyl-2-cyano-pyrrolidine, LAF237 or vildagliptin (INN)) of formula

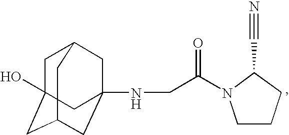

and L-threo-isoleucyl thiazolidine (compound code according to Probiodrug: P32/98 as described above), MK-0431, GSK23A, saxagliptin, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally in any case pharmaceutical salts thereof.

DPP728 and LAF237 are specifically disclosed in Example 3 of WO 98/19998 and Example 1 of WO 00/34241, respectively. DPP728 and LAF237 can be formulated as described on page 20 of WO 98/19998 or in WO 00/34241, or in the International Patent Application No. EP2005/000400 (application number).

Any of the substances disclosed in the above mentioned patent documents or scientific publications, hereby included by reference, are considered potentially useful as DPP-IV inhibitors to be used in carrying out the present invention.

DPP-IV inhibitor to be used alone according to the present invention can be used in association with a carrier.

A carrier in the instant context is a tool (natural, synthetic, peptidic, non-peptidic) for example a protein which transports specific substances through the cell membrane in which it is embedded and into the cell. Different carriers (natural, synthetic, peptidic, non-peptidic) are required to transport different substances, as each one is designed to recognize only one substance, or group of similar substances.

Any means of detection known by the person skilled in the art can be used to detect the association of the DPP-IV with a carrier, for example, by labelling the carrier.

Most preferred are orally active DPP-IV inhibitors and pharmaceutical salts thereof.

The active ingredients (metformin or DPP-IV inhibitors) or pharmaceutically acceptable salts thereof according to the present invention may also be used in form of a solvate, such as a hydrate or including other solvents, used for crystallization.

In the present context the terms “(S)-1-[(3-hydroxy-1-adamantyl)amino]acetyl-2-cyano-pyrrolidine” or “LAF237” or “vildagliptin” is also intended to comprise any salt or crystal form thereof.

In a most preferred embodiment the DPP-IV inhibitor is added to the standard diabetes treatment in patients whose disease was not adequately controlled by metformin alone.

The present methods or uses are particularly useful for the prevention or delay of progression of conditions associated with type 2 diabetes or IGT, particularly cardiovascular and microvascular conditions.

Metformin, i.e. N,N-dimethylimidocarbonimide diamide, is a known compound approved by the U.S. Food & Drug Administration for the therapeutic treatment of diabetes. The compound and its preparation are disclosed, for example, in U.S. Pat. No. 3,174,901, issued May 23, 1965.

In the present context the term “metformin” is also intended to comprise any salt or crystal form, especially the metformin hydrochloride salt.

The term “before the evening meal” used herein means administration of the DPP-4 inhibitor up to 1 hours, preferably up to 30 minutes before the evening meal e.g. 10, 5 or 1 minute before the evening meal.

The term “with the evening meal” used herein means administration of the DPP-4 inhibitor concomitantly with evening meal or optionally up to e.g. 10, 5 or 1 minute after the evening meal.

Furthermore as used herein, “a daily dose” means the dose given within a 24-hour period.

The term “prevention” means prophylactic administration of the active ingredient to healthy patients to prevent the outbreak of the conditions mentioned herein. Moreover, the term “prevention” means prophylactic administration of such active ingredient to patients being in a pre-stage of the conditions, to be treated.

The term “delay of progression” used herein means administration of the active ingredient, to patients being in a pre-stage of the condition to be treated in which patients a pre-form of the corresponding condition is diagnosed.

By the term “treatment” is understood the management and care of a patient for the purpose of combating the disease, condition, or disorder. The term “treatment” also covers the “delay of progression” of the treated disease.

As used herein, the term “patient” refers to an animal who is suffering from hyperglycemia e.g. type 2 diabetes or IGM. The preferred animal is a mammal, such as dogs, cats, horses, cows and humans. It is preferred that the patient is a human.

In this field the preferred patient population age is from 45 years onwards.

The term “a patient with type 2 diabetes and with baseline HbA1c>8.0%” refers to a patient having a glycosylated hemoglobin level i.e. HbA1c level higher than 8%, prior to the treatment following the herein described method of treatment.

The person skilled in the pertinent art is fully enabled to select a relevant test model and protocols to prove the beneficial effects of the invention.

Monitoring of glycemic status, as performed by patients and health care providers, is well known in the art such as reported in Diabetes Care “Tests of Glycemia in Diabetes—American Diabetes Association” 2003 26: S106-108 and described below. This publication is hereby incorporated by reference in their entirety.

The American Diabetes Association's technical review should be consulted forfurther information (e.g. Goldstein D E, Little R R, Lorenz R A, Malone J I, Nathan D, Peterson C M: Tests of glycemia in diabetes (Technical Review). Diabetes Care 18:896-909, 1995).

### Glycated Hemoglobin (GHb or HbA_{1c}) Testing:

GHb, also referred to as glycohemoglobin, glycosylated hemoglobin, HbA_{1c}, or HbA₁, is a term used to describe a series of stable minor hemoglobin components formed slowly and nonenzymatically from hemoglobin and glucose. The rate of formation of GHb is directly proportional to the ambient glucose concentration. Since erythrocytes are freely permeable to glucose, the level of GHb in a blood sample provides a glycemic history of the previous 120 days, the average erythrocyte life span.

Many different types of GHb assay methods are available to the routine clinical laboratory e.g. HbA_{1c} can be measured by High Performance Liquid Chromatography (HPLC) using the ion-exchange method on a Bio-Rad Diamat analyzer.

Methods differ considerably with respect to the glycated components measured, interferences, and nondiabetic range. HbA_{1c} has become the preferred standard for assessing glycemic control. In referring to this test, the term “A1C test” will be used.

The glucose level progression checks (e.g. GSP assay, A1C, insulin) are well known by the physicians and reported in the art e.g. by the American Diabetes Association.

Preferably the treated patient in the above described methods or uses, is suffering from hyperglycemia. Most preferably the patient suffering from hyperglycemia, is suffering from a disease selected from diabetes mellitus, type I diabetes, type 2 diabetes, type A insulin resistance, IGM, IFG or IGT. In a preferred embodiment the patient is suffering from type II diabetes or IGT. In another preferred embodiment the treated patient is a patient whose disease was not adequately controlled by metformin alone.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

Preferably the active ingredients (e.g. DPP-4 inhibitor) are comprised in a pharmaceutical preparation (pharmaceutically acceptable carriers). The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carries, especially suitable for enteral or parenteral application.

Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubulizing or lyophilising processes. Thus, pharmaceutical preparations for oral use can be obtained by combining the active compound with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

The dosage of the active compound can depend on a variety of factors, such as mode of administration, homeothermic species, age and/or individual condition.

The corresponding active ingredient or a pharmaceutically acceptable salt thereof may also be used in form of a hydrate or include other solvents used for crystallization.

The exact dosage will of course vary depending upon the compound employed, mode of administration and treatment desired. The compound may be administered by any conventional route, non-oral or preferably orally.

In general, satisfactory results are obtained when DPP-IV inhibitor especially vildagliptin is administered at a daily dosage of from about 0.01 to 50 mg/kg, more preferred doses ranged from 0.1 to 50 mg/kg.

For the larger mammals, an indicated total daily dosage is in the range from about 0.01 to 100 mg/kg of the compound, conveniently administered in divided doses 2 to 4 times a day in unit dosage form containing for example from about 0.1 to about 50 mg of the compound in sustained release form.

Preferably for the DPP-IV inhibitor especially vildagliptin an indicated total daily dosage is in the range from between 1 and 500 mg, preferably between 10 and 200 mg of active ingredient.

The daily oral dosage of vildagliptin is between 1 and 200 mg preferably between 10 and 200 mg e.g. 10 mg, most preferably between 25 and 100 mg e.g. 25 mg or 30 or 40 or 50, 61, 70, 90, 100 mg. The very preferred daily oral dosage of vildagliptin is between 50 and 100 mg.

Appropriate unit doses for oral administration contain for example about 25 to about 100 mg of DPP-IV inhibitor especially vildagliptin, such as preferably 25, 50 or 100 mg. Appropriate doses for parenteral administration contain for example about 1 to about 100 mg of the compound, e.g. from 10 to 50 mg.

The DPP-IV inhibitor can also be administered e.g. every day or twice a day.

The compounds may be administered in similar manner to known standards for uses in these utilities. The suitable daily dosage for a particular compound will depend on a number of factors such as its relative potency of activity. A person skilled in the pertinent art is fully enabled to determine the therapeutically effective dosage.

The compound of the invention may be administered in free base for or as a pharmaceutically acceptable acid addition or quaternary ammonium salt. Such salts may be prepared in conventional manner and exhibit the same order of activity as the free forms. If these compounds have, for example, at least one basic center, they can form acid addition salts. Corresponding acid addition salts can also be formed having, if desired, an additionally present basic center. The compounds having an acid group (for example COOH) can also form salts with bases

In the present invention the DPP-IV inhibitor can also be in the form of a combination which comprises a DPP-IV inhibitor in free or pharmaceutically acceptable salt form, and metformin or the pharmaceutically acceptable salt thereof and optionally at least one pharmaceutically acceptable carrier; wherein the active ingredients can be administered simultaneously or sequentially in any order, separately or in a fixed combination (same galenic formulation).

A combined preparation which comprises a DPP-IV inhibitor in free or pharmaceutically acceptable salt form and metformin or a pharmaceutically acceptable salt thereof, and optionally at least one, i.e., one or more, e.g. two, pharmaceutically acceptable carrier for simultaneous, separate or sequential use is especially a “kit of parts” in the sense that the components, a DPP-IV inhibitor in free or pharmaceutically acceptable salt form and metformin or the pharmaceutically acceptable salt thereof, can be dosed independently or by use of different fixed combinations with distinguished amounts of the components, i.e. at different time points or simultaneously. The parts of the kit of parts can then, e.g., be administered simultaneously or chronologically staggered, that is at different time points and with equal or different time intervals for any part of the kit of parts.

A therapeutically effective amount of each of the components of the combination of the present invention may be administered simultaneously or sequentially and in any order, and the components may be administered separately or as a fixed combination.

The invention has been described above by reference to preferred embodiments but, as those skilled in the art will appreciate, many additions, omissions and modifications are possible all within the scope of the claims below.

All patents and literature references cited in this specification are hereby incorporated by reference in their entirety. In case of inconsistencies, the present description, including the definitions and interpretations, will prevail.

### EXAMPLE 1

### Clinical Study—Study Synopsis and Assessment Schedule

### Investigational Drug:

1) Vildagliptin: 100 mg tablet-2) Matching placebo tablet

### Title of Study:

A randomized, double-blind, two period, crossover study to compare glycemic profiles in patients with type 2 diabetes after administration of 100 mg vildagliptin once daily in the morning or the evening.

### Objectives:

Basal hepatic glucose production (HGP) in type 2 diabetic patients is elevated and the increase in HGP correlates closely with the severity of fasting hyperglycemia. The objective of this study is to explore whether or not the dosing regimen given in the evening (e.g. as once daily) can result in better suppression of HGP during the overnight, and hence result in better control of the 24 hour glycemic profiles in addition to FPG (fasting plasma gluscose).

### Primary Objective:

To compare the effect of an oral dosing of 100 mg vildagliptin administered once daily in the morning versus evening on postprandial glucose profiles over 24 hours after 28 days of treatment

### Secondary Objective(s):

To compare the effect of 100 mg vildagliptin given orally in either the morning or the evening on fasting plasma glucose (FPG) levels after treatment for 28 days.
To compare the effect of 100 mg vildagliptin given orally in either the morning or the evening on DDP4, GLP-1, glucagon, and insulin levels.
To evaluate the safety and tolerability of 100 mg vildagliptin given once daily in the evening.
   Design: This is a randomized, double-blind, two-period, crossover study. A total of 40 patients with type 2 diabetes (male and female) are randomized and at least 38 complete the study (if the number of patients continuing in the study falls to 38, any further discontinued patients must be replaced). Eligible patients participate in a two day single-blind placebo treatment (run-in period). After which, each patient is randomized to receive the following two treatments (AB or BA):
   Treatment A: one 100 mg vildagliptin tablet in the morning, one placebo tablet in the evening (28 days)
   Treatment B: one placebo tablet in the morning; one 100 mg vildagliptin tablet in the evening (28 days)

There is a 28-day screening period including a 21-day washout from prior oral hypoglycemic agents such as metformin and sulfonylurea. A fasting plasma glucose (FPG) sample is drawn from each patient at screening, Day −7 (during washout), and at baseline (Day −3). Patients that meet inclusion/exclusion criteria are admitted to the study center on the evening of Day −4. On Day −3 (following 10-h fast), baseline assessments are completed to confirm patient eligibility.

On Day −2, patients begin the single-blind placebo run-in period. Each patient is given placebo tablets bid for two days (Day −2 & Day −1): one tablet in the morning 30 minutes prior to breakfast and one in the evening 30 minutes prior to dinner. Whenever domiciled, patients consume standardized meals: breakfast beginning at approximately 08:00, lunch beginning at approximately 12:00, and dinner beginning at approximately 18:00. On Day −1, the morning dose is administered following an overnight (10 h) fast. Patients consume a standard ADA breakfast 30 minutes postdose and blood sampling occurs at specified times for glucose, insulin, DPP-4, GLP-1, and glucagon up to 24-hr post morning dose. Following this run-in period, eligible patients participate in two consecutive 28-day treatment periods (out-patient) and an end of study evaluation. The run-in period is single blind; whereas the two 28-day treatment periods is double blind.

On Day 1, the first dose of the first treatment period is administered following an overnight fast (at least 10 h). Patients consume a standard ADA breakfast 30 minutes following the drug administration. Patients are discharged from the study center 4 hours postdose with guidance for dietary maintenance, adherence to previous exercise regimen, out-patient medication for 28 days, instructions for outpatient dose administrations, a glucometer, an out-patient diary, and a calendar marked with scheduled return visits. Each patient receives 2 study medication bottles-one contains medication which will only be taken in the morning and the other bottle contains study medication that will only be taken in the evening. During the out-patient days, patients administer the morning and evening doses immediately prior to breakfast and dinner meals, respectively. Patients return to the study center for an interim safety assessment, review of out-patient diaries, and pill-count once on Day 14 of each Treatment Period. Patient compliance is assessed on these visits through drug accountability. Patients who have missed more than 10% (6 doses) of study medication is not allowed to continue in the study.

For pharmacokinetic and pharmacodynamic assessments, patients return to the study center on the evening of Day 26 of each Treatment Period and will remain domiciled through the morning of Day 29. On Day 26, patients self administer the evening dose and eat dinner prior to arrival at study site. On Day 28 of each Treatment Period, the morning dose is administered following an overnight (10 h) fast. Patients have to consume a standard ADA breakfast (at 08:00) 30 minutes after dosing (at 07:30) and blood sampling occurs at specified times for vildagliptin, DPP-4, GLP-1, glucose, insulin, and glucagon up to 24-hr post morning dose. Of note, predose glucose levels on Day 28 of each Treatment Period is used to determine FPG. Samples for HbA1c measurement are collected prior to dosing on Day 28 of each treatment period. The evening dose is administered 30 minutes prior to the dinner meal (meal at 18:00).

Based on the results of safety assessments on Day 27 of Treatment Period 1, patients that continue to meet eligibility criteria begin dosing for Treatment Period 2 a day after the conclusion of PD sampling for Period 1.

Study completion evaluations occur following the last pharmacokinetic/pharmacodynamic assessment in Treatment Period 2 (Day 29). Table 1 provides an overview of the study.

TABLE 1
Study Overview

| Study Section | Day(s) | Study requirements |
|---|---|---|
| Screening | Days −32 to −5 | HbA1c levels is determined at screening |
| | | FPG is determined at screening and Day −7 |
| | | Screening assessments: |
| | | Medical history, concomitant medications, physical exam, |
| | | vitals, ECG, safety labs, pregnancy test, |
| | | inclusion/exclusion criteria |
| | | 21 day washout from prior hypoglycemic agents |
| Baseline/Run- | Day −4 to −1 | Patients admitted in the evening of Day −4. |
| in Period | | Day −3 baseline assessments are conducted: |
| | | Confirm inclusion/ exclusion |
| | | FPG |
| | | Day −2 & −1: Patients given placebo tablets (bid) |
| | | Day −1 (following 10 h fast): PD assessments over 24 h |
| Treatment | Days 1-13 | Complete PD assessments |
| Period 1 | | Day 1: Morning dose after 10 hr fast; patients given |
| | | standardized breakfast 30 min after dosing. Patients |
| | | released 4 hours after dosing |
| | | Receive drug, diary and instructions |
| | | Out-patient dosing for remaining study days (Day 1, |
| | | evening dose through Day 26 evening dose) |
| | Day 14 | Patients return to study center |
| | | Safety assessment |
| | | Drug accountability and diary review |
| | Days 15-26 | Out-patient dosing |
| | | Patients return to study center on the evening of Day 26 |
| | | (patients administer evening dose and eat dinner prior to |
| | | arrival at study site) |
| | Day 27 | Patients given standardized meals |
| | | Safety assessments |
| | | Drug accountability and diary review |
| | Day 28 | Morning dose after 10 h fast |
| | | Patients given standardized breakfast 30 min after dosing |
| | | PK/PD assessments over 24 hours |
| | Day 29 | PK/PD sampling for period 1 concludes |
| | | Following last PK/PD sample, patients are discharged with |
| | | medication, diary, and instructions for the next treatment |
| | | period* |
| Treatment | Day 1-13 | Out-patient dosing. |
| Period 2 | Day 14 | Patients return to study center |
| | | Safety assessment |
| | | Drug accountability and diary review |
| | Days 15-26 | Out-patient dosing |
| | | Patients return to study center on the evening of Day 26 |
| | Days 27 | Patients given standardized meals |
| | | Safety assessments |
| | | Drug accountability and diary review |
| | Day 28 | Morning dose after 10 h fast |
| | | Patients given standardized breakfast 30 min after dosing |
| | | PK/PD assessments over 24 hours |
| EOS | Day 29 | End of study evaluations begin following the last PK/PD |
| | | sample |
| | | Patients are released form study center following end of |
| | | study evaluations. |

*Only patients that continue to meet inclusion criteria following safety assessments on Day 27 are provided medication for Treatment Period 2 at the time of discharge.

### Number of Patients: 40 (38 Must Complete all Assessments)

### Summary of Criteria for Inclusion:

Male or female (post-menopausal, surgically sterile, or using double barrier method of contraception)
Aged 18-75 years with type 2 diabetes
Patients must be diagnosed with type 2 diabetes for at least 3 months. Patients must be otherwise in good health as determined by past medical history, physical examination, electrocardiogram, laboratory tests and urinalysis
Patients whose diabetes is controlled by diet and exercise alone or are willing to undergo a 3 week hypoglycemic washout.
Fasting plasma glucose between 110-240 mg/dL (6.0-13 mmol/L) at screening and baseline
HbA_{1c} from 7.0-10% at screening
Body mass index ≦35 kg/m²
Patients must be willing to comply with all study requirements and provide written informed consent

### Duration of Treatment:

58 days (2-day run-in period plus two 28-day treatment periods)

### Pharmacokinetic Assessments:

Blood collection for vildagliptin determination (1 mL blood Per sample, heparin tubes (plasma)):
   Day 28 (Period 1) and Day 28 (period 2):
   Predose, 0.5, 1, 1.5, 2, 3, 5, 7, 10 (before evening dose), 10.5, 11, 11.5, 12, 13, 15, 17, and 24 h post morning dose
Analytes, media and methods: LAF237 in plasma by LC-MS/MS; LOQ of approximately 2 ng/mL
PK parameters for vildagliptin: Cₘₐₓ, tₘₐₓ, AUC_{0-∞}, AUC₀₋ₜ, t_{1/2}

### Pharmacodynamic Assessments:

1. Plasma DPP-4 activity (1 mL blood sample)
Day −1 (2^{nd} day of Run-in placebo treatment), Day 28 (period 1) and Day 28 (period 2):
   Predose, 0.50, 0.75, 1, 1.25, 1.5, 2.5, 5.5, 10.5 (pre evening meal), 10.75, 11, 11.5, 12.5, 15.5, and 24 hr post morning dose
      2. Plasma GLP-1 (2 mL blood sample)
Day −1 (2^{nd} day of Run-in placebo treatment), Day 28 (period 1) and Day 28 (period 2):
   Predose, 0.583, 0.666, 0.75, 1, 1.25, 1.5, 2, 2.5, 3.5, 5.5, 8.5, 10.5 (pre-dinner meal), 10.583, 10.666, 10.75, 11, 11.25, 11.5, 12, 12.5, 14.5, 17.5 and 24 hr post morning dose
      3. Glucagon (2 ml blood per sample)
Day −1 (2^{nd} day of Run-in placebo treatment), Day 28 (period 1) and Day 28 (period 2):
   Predose, 0.583, 0.666, 0.75, 1, 1.25, 1.5, 2, 2.5, 3.5, 5.5, 8.5, 10.5 (pre-dinner meal), 10.583, 10.666, 10.75, 11, 11.25, 11.5, 12, 12.5, 14.5, 17.5 and 24 hr post morning dose
      4. Insulin & glucose (2.5 mL blood per sample)
Day −1 (2^{nd} day of Run-in placebo treatment), Day 28 (period 1) and Day 28 (period 2):
   Predose*, 0.5, 1, 1.25, 1.5, 1.75, 2, 2.5, 3, 3.5, 4.5 (pre-lunch meal), 5, 5.25, 5.5, 6, 6.5, 7, 7.5, 8.5, 10.5 (prior to evening dose and pre-dinner meal), 11, 11.25, 11.5, 11.75, 12, 12.5, 13, 13.5, 14.5, 17.5, 24 hr post morning dose.
   *Predose samples on Day 28 of each Treatment Period are also used to determine fasting plasma glucose Estimated total blood volume taken per patient: 672 mL

### Statistical Methods

1. Sample size: Sample size for this study is determined based on one sample two-sided t-test at 5% significance level, considering the crossover design is employed and the underlying null hypothesis is to test for equality of means against un-equality for the primary PD endpoint (glucose AUE(0-24)). Because of the lack of variability information about the PD endpoint, a range of variability inputs is evaluated to provide information for the required sample size for the current study. A previous study using BID dosing regimen showed the intrapatient coefficient of variation for glucose AUE(0-14) was approximately equal to 0.20. It is assumed that the variability of AUE(0-24) is larger than that of AUE(0-14). When an intrapatient CV is not greater than 0.30, a sample size of 40 patients will ensure at least 87% power to detect a 15% difference between AM and PM dosing regimens (see Table 2).

TABLE 2
Statistical power to detect the true difference under
the given variability for sample size of 40 patients

| True difference | | |
|---|---|---|
| (in % of mean from AM | Intrapatient CV | Power |
| regimen) | (%) | (%) |
| 10 | 20 | 87 |
| | 25 | 69 |
| | 30 | 54 |
| 15 | 20 | 99 |
| | 25 | 96 |
| | 30 | 87 |
| 20 | 20 | 99 |
| | 25 | 99 |
| | 30 | 98 |

2. Statistical analysis: An analysis of variance (ANOVA) is performed on log-transformed PD variables using the PROC MIXED SAS procedure. The sources of variation included in the ANOVA model is sequence, patient (sequence), period, and treatment, with patient (sequence) as random effect. The AM dosing is the test treatment and PM dosing the reference. Using the ESTIMATE statement of the PROC MIXED SAS procedure, the contrast is constructed between the test and the reference treatments to obtain the p-value, the estimated mean difference, and the 95% confidence interval (CI) for the log-scale test-reference difference. The anti-logs of estimated mean difference and the 95% CI constitute the ratio of geometric means and the 95% CI for the true test-reference ratio. The outputs from the comparison is tabulated.

### Pharmacokinetic Assessments:

Blood collection: All blood samples will be taken by either direct venipuncture or an indwelling cannula inserted in a forearm vein. For each scheduled LAF237 (vildagliptin) sample, collect a 1 mL blood sample into a sodium or lithium heparin tube. Samples will be collected according to the pre-defined schedule presented in the Study Synopsis.
Handling of blood samples: Immediately after each tube of blood is drawn, it should be inverted gently several times to insure the mixing of tube contents (e.g., anticoagulant). Avoid prolonged sample contact with the rubber stopper. Place the tube upright in a test tube rack (e.g. surrounded by ice or at room temperature) until centrifugation. Within 15 minutes, centrifuge the sample between 3 and 5° C. for 15 minutes at approximately 2500 rpm. Transfer all available plasma to a polypropylene screw-cap tube and freeze at −70° C. or below within 60 minutes of venipuncture.
Analytical method(s): Analytes, media and methods: vildagliptin in plasma by LC-MS-MS; LOQ at 2 ng/mL.

### Pharmacodynamic Assessments:

Samples will be collected according to the pre-defined schedule presented in the Study Synopsis.

The study site must provide the actual date and time of the collection with the sample and must document on the appropriate CRF. A separate document outlining procedures for sample collection, processing and shipping will be supplied as a Protocol supplement.
Glucose, Insulin, and Glucagon samples: Plasma glucose, insulin, and glucagon are measured. For each scheduled sample collection for glucose and insulin analysis, collect a 2.5 ml blood sample into a tube containing Heparin (e.g. BD 367960—3 mL PST, heparin plasma separator tubes). Place the tube upright in a rack surrounded by ice until centrifugation. Within 15 minutes of collection, centrifuge the sample at between 3 and 5° C. for 15 minutes at approximately 2500 rpm. Transfer all available plasma (at least 1.0 mL) to a polypropylene screw-cap tube and freeze at −80° C. or below within 60 minutes of venipuncture. For each scheduled sample collection for glucagon analysis, collect a 2 mL blood sample into an iced glass tube containing Aprotinin. Invert each tube gently several times to mix the contents of the tube, avoiding prolonged sample contact with the rubber stopper. Place the tube upright in rack surrounded by ice until centrifugation. Within 15 minutes of collection, centrifuge the sample between 3 and 5° C. for 15 minutes at approximately 2500 rpm. Using a glass pipette, transfer all available plasma (at least 0.75 mL total) to a CRO supplied glass transfer tube and freeze at −80° C. or below within 60 minutes of venipuncture. A separate document outlining the collection and handling of the PD samples will be supplied.
GLP-1 plasma level: For each scheduled sample collection for GLP-1 analysis, collect a 2 ml blood sample into a tube containing potassium EDTA to which 0.1-ml of a 3 mM Diprotin A solution has been added. Invert each tube gently several times to mix the contents of the tube, avoiding prolonged sample contact with the rubber stopper. Place the tube upright in rack surrounded by ice until centrifugation. Within 15 minutes of collection, centrifuge the sample between 3 and 5° C. for 15 minutes at approximately 2500 rpm. Transfer all available plasma (should approximate 1.3 ml total) to a polypropylene screw-cap tube and freeze at −70° C. or below within 60 minutes of venipuncture.
DPP-4 enzyme inhibition plasma samples: For each scheduled sample collection for DPP-IV enzymatic analysis, collect a 1 ml blood sample into a tube containing potassium EDTA. Invert gently several times to mix the contents of the tube. Avoid prolonged sample contact with the rubber stopper. Place the tube upright in rack surrounded by ice until centrifugation. Within 15 minutes after collection, centrifuge the sample between 3 and 5° C. for 15 mins at approximately 2500 rpm. Transfer all available plasma to a polypropylene screw-cap micro tube and freeze at −70° C. or below within 60 minutes of venipuncture.
HbA1c: HbA1c are measured by the clinical laboratory associated with the study site, concurrent with the blood chemistry analysis at screening (same blood sample may be used). Additional blood samples for analysis of HbA1c are taken prior to dosing on Day 28 of each Treatment Period.
Fasting Plasma Glucose (FPG): Fasting blood glucose levels are performed at the clinical laboratory associated with the study site during screening, Day −7 (during washout), and at baseline (Day −3). Of note, predose glucose levels on Day 28 of each Treatment Period are used to determine FPG.

### Pharmacodynamic Data Analysis:

Pharmacodynamic variables: All patients who complete the trial with evaluable pharmacodynamic (PD) assessments are included in the data analysis. Pharmacodynamic responses are explored by graphical representations.

Plasma DPP-4, GLP1, glucagon, glucose, and insulin levels are measured as the pharmacodynamic variables. The plasma concentrations of these variables are plotted versus time and the pharmacodynamic response are explored by inspecting the graphical representations. As appropriate, the change from predose value or baseline are calculated for each subject at each time point. Percentage inhibition of DPP-4 activity are calculated and plotted over time for each subject during each treatment period. Areas under the effect vs time curves (AUE) for GLP-1, glucagon, glucose, and insulin are calculated when deemed necessary. Peak effect (Eₘₐₓ) and time to the peak effect (Tₘₐₓ) for each PD variable are assessed when deemed appropriate.
Statistical methods for pharmacodynamic analysis: An analysis of variance (ANOVA) are performed on log-transformed PD variables using the PROC MIXED SAS procedure. The sources of variation included in the ANOVA model are sequence, patient (sequence), period, and treatment, with patient (sequence) as random effect. The AM dosing is the test treatment and PM dosing the reference. Using the ESTIMATE statement of the PROC MIXED SAS procedure, the contrast is constructed between the test and the reference treatments to obtain the p-value, the estimated mean difference, and the 95% confidence interval (CI) for the log-scale test-reference difference. The anti-logs of estimated mean difference and the 95% CI constitute the ratio of geometric means and the 95% CI for the true test-reference ratio. The outputs from the comparison are tabulated.

### Results:

The administration of vildagliptin before or with the evening meal provides additional pharmacokinetic and especially pharmacodynamic benefits. Especially the resulting data show an unexpected additional benefit on the overnight HGP reduction, especially in patients with more severe type 2 diabetes i.e. with baseline glycosylated hemoglobin (HbA1c)>8.0%.

1. A method for treating a patient suffering from hyperglycemia, wherein vildagliptin or a salt thereof is administered to said patient before or with the evening meal.

2. (canceled)

3. A method to reduce the overnight hepatic glucose production in a patient suffering from hyperglycemia, wherein vildagliptin or a salt thereof is administered to said patient before or with the evening meal.

4. (canceled)

5. A method according to claim 1 or 3, wherein the patient is suffering from type 2 diabetes.

6. A method according to claim 1 or 3, wherein the patient is suffering from type 2 diabetes with baseline HbA1c>8.0%.

7. A method according to claim 1 or 3, wherein between 25 mg and 100 mg or between 50 mg and 100 mg of vildagliptin or a salt thereof, is administered before or with the evening meal.

8. A method according to claim 1 or 3, wherein 25 mg, 50 mg or 100 mg of vildagliptin or a salt thereof, is administered before or with the evening meal.

9. A method according to claim 1 or 3, wherein a daily dosage of 100 mg of vildagliptin or a salt thereof, is administered to the patient.

10. A method to reduce the overnight hepatic glucose production in a patient suffering from hyperglycemia, wherein at least 100 mg of vildagliptin or a salt thereof is administered to said patient before or with the morning meal, or before or with the lunch.

11. (canceled)

12. A method according to claim 10, wherein the patient is suffering from type 2 diabetes.

13. A method according to claim 12, wherein the patient is suffering from type 2 diabetes with baseline HbA1c>8.0%.

14. A method according to claim 10, 12, or 13, wherein between 100 and 150 mg of vildagliptin or a salt thereof is administered to said patient before or with the morning meal, or before or with the lunch.

15. A method according to claim 10, 12, or 13, wherein 100 mg or 150 mg of vildagliptin or a salt thereof is administered to said patient before or with the morning meal, or before or with the lunch.

16. A method according to claim 10, 12, 13 or 15, wherein vildagliptin or a salt thereof is administered in combination with metformin.

17. A method according to claim 10, 12, 13, 15 or 16, wherein vildagliptin or a salt thereof is administered in combination with 250 mg, 500 mg, 750 mg, 850 mg or 1000 mg of metformin.

18. A method according to claim 10, 12, 13, 15, 16 or 17, wherein metformin is administered with the evening meal.

19. A method according to any of the previous claims wherein vildagliptin or a salt thereof is administered about 30 minutes prior to the dinner meal.
